(19) United States
(12) Patent Application Publication
(10) Pub. No.: US 2007/0154550 A1
(43) Pub. Date: Jul. 5, 2007

(54) Pharmaceutical composition comprising anticonvulsant with taste mask coating

(76) Inventor: POTDAR ARTI, Maharashtra (IN)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/570,216

(22) PCT Filed: Aug. 27, 2004
(86) PCT No.: PCT/EP04/09588

§ 371 Date: Dec. 5, 2006

(30) Foreign Application Priority Data
Aug. 28, 2003 (EP) 03468006.6

(51) Int. Cl. A61K 9/24

(52) U.S. Cl. 424/472

(57) ABSTRACT

A pharmaceutical composition in the form of coated core particles comprising an anticonvulsant drug, which can be sprinkled onto food, wherein said core particles are coated with a taste mask coating which is less than 7% by weight of the pharmaceutical composition.

### FIELD OF THE INVENTION

This invention relates to a solid dosage pharmaceutical composition and a process for preparing said solid dosage pharmaceutical composition. More particularly, the solid dosage pharmaceutical composition refers to coated core particles comprising an anti-convulsant drug, which can be sprinkled onto food to ease administration. Most particularly the solid dosage formulation consists of core particles which are sugar beads or spheres or pellets coated with topiramrate and a polymeric outer coating, which serves to stabilize the core particles and to mask the bitter taste of active ingredient.

### BACKGROUND OF THE INVENTION

Topiramate is a 2,3:4,5-bis-O-(1-methylethylidene)-β-D-fructopyranose sulfamate first disclosed in EP 138441. It is used as an anti-convulsant drug for the treatment of epilepsy.

EP 1066027 claims a process for producing a pharmaceutical composition characterised by preparing core particles comprising as active agent topiramate; drying the core particles from first step to form dried core particles; coating the dried core particles from previous step with a taste masking mixture to form coated particles; and drying the coated particles to form the pharmaceutical composition wherein the amount of taste masking mixture ranges from about 7% by weight to about 15% by weight of the pharmaceutical composition.

Making pleasant tasting pharmaceutical compositions demands a subtle balance of taste masking and bloavailability properties. The authors of EP 1066027 achieved that by applying a taste masking coat of about 11% by weight of a pharmaceutical composition when dried.

EP 181515 claims a process for preparing a coating agent dispersion for medicaments which forms a film on drying, by dispersing a powder coating agent in an aqueous phase with stirring at elevated temperature, characterized in that a powdery copolymer (including e.g. aminoalkyl methacrylate) which is swellable, but insoluble, in water, is dispersed in the aqueous phase.

EP 302900 claims a taste masking pharmaceutical composition with a core comprising a pharmaceutically active agent and a polymer mixture coating said core, characterized by said mixture comprising a high temperature film forming polymer and a low temperature film forming polymer.

Those documents, among others, also show the importance of physical properties of the coating, such as integrity (i.e. the coating does not fracture and release active ingredient when tabletted and/or chewed), swelling, cracking and elasticity of the coating. J. Pharm. Biomed. Anal. 29 (2002), pages 69-74, discloses an analytical method which can assess the integrity of coated pharmaceuticals, and which can indirectly measure the completeness of taste masking in the sprinkle formulation.

Topiramate is susceptible to heat and moisture. Upon exposure to heat and moisture inherently some degradation of the active ingredient occurs. Degradation of topiramate is readily detected by changes in physical appearance i.e. discoloration to brown or black, and formation of sulphate ions, which can be measured by standard techniques.

A typical solution to overcome this problem is to avoid water or moisture as shown in EP 1157682, where a blister package for topiramate tablets containing less than 1,4% free water and a process for its preparation are disclosed.

It is also known to a person skilled in the art to protect the active ingredient by applying a coating which diminishes the contact of the outside environment with the active ingredient, and in the case of moisture sensitive and poorly stable active ingredients such as topiramate to use volatile organic solvents while applying the coating. However, organic solvents are not preferred in pharmaceutical processes due to ecological reasons and pharmacopoeia requirements. Thus there is a need to find alternatives to the use of organic solvents for applying the above mentioned coating.

On the other hand, topiramate is known to have an extremely bitter taste, which is disadvantageous with special solid pharmaceutical forms such as particles, which can be sprinkled onto food and swallowed therewith. These forms are often desirable for patients who have difficulties to swallow conventional dosage forms such as tablets and/or capsules as a whole, for example for children or older persons. A major requirement of any such solid form is that it must be palatable to reduce the risk of a patient neglecting to take the medication. In cases where the active ingredient is particularly unpalatable and somewhat unstable, such as topiramate, it is difficult to prepare such solid forms which fulfill said requirement, and in addition show good stability and bloavailability.

Accordingly, it is a goal of the present invention to develop a pharmaceutical composition of topiramate which is both stable and bloavailable, and which has a coating with satisfactory taste-masking properties, for use in children and patients who have difficulty in swallowing conventional solid forms (e.g. tablets or capsules). It is a further goal of the invention to find an environment-friendly alternative to the organic solvents used in the coating process.

### SUMMARY OF THE INVENTION

One aspect of the invention is the preparation of a stable solid pharmaceutical composition comprising preferably at least one drug which is sensitive to moisture and/or heat, and/or which has an unpleasant and/or bitter taste. More preferably, said drug is an anti-convulsant drug, most preferably, topiramate. In particular, the solid dosage pharmaceutical composition refers to coated core particles comprising an anti-convulsant drug, preferably topiramate. Most preferably, the pharmaceutical composition is in the form of sprinkle capsules, which can ease administration to patients who have difficulty swallowing tablets or capsules, e.g. pediatric patients.

Accordingly, the present invention provides a pharmaceutical composition comprising
(a) core partides comprising a therapeutically effective amount of at least one active ingredient and optionally at least one excipient, and
(b) a taste mask coating, wherein the amount of taste mask coating is less than 7% by weight of the pharmaceutical composition.

Preferably, the active ingredient has a particle size of up to 50 microns μm).

Preferably, the active ingredient is sensitive to heat and/or moisture and/or unpalatable, more preferably the active ingredient is an anticonvulsant drug, most preferably topiramate.

Preferably, the amount of taste mask coating is less than 6%, or may range from about 1% to about 5% by weight of the pharmaceutical composition.

The essence of the invention is to coat the drug loaded cores with water-dispersible and/or water-soluble polymers instead of using a coating agent which is dissolved, suspended or dispersed in organic solvents. Drug loaded cores are understood to mean core particles comprising at least one excipient with a pharmaceutically active ingredient disposed thereon or therein.

Another aspect of the invention is the use of an aqueous polymer mixture for coating of the core particles. Preferably, the aqueous polymer mixture is a taste masking mixture. The term “aqueous polymer mixture” as used herein is understood to mean a mixture which comprises a water-dispersible and/or water-soluble polymer or copolymer. If the water-dispersible and/or water-soluble polymer used for the coating is selected among aminoalkyl methacrylate polymer or copolymer or ethyl cellulose, preferably aminoalkyl methacrylate polymer or copolymer, mixed with a sufficiently small amount of water, a stable pharmaceutical composition comprising topiramate is achieved in spite of the drug being sensitive to moisture. A sufficiently small amount of water means that said amount is essentially removed during the process of drying of the composition, so that said composition is essentially free of water.

Another aspect of the invention provides the preparation of a stable pharmaceutical composition comprising at least one active ingredient as herein described, preferably topiramate, using an aqueous coating technique applying a water-dispersible and/or water-soluble coating agent, e.g. a taste masking agent, in an amount ranging from about 1% by weight to about 5% by weight of the pharmaceutical composition.

In a further aspect, the present invention provides a process for preparing a pharmaceutical composition comprising the following steps:
(a) preparing core partides comprising at least one active ingredient;
(b) drying the core particles obtained in step (a) to form dried core partides;
(c) coating the dried core particles obtained in step (b) with an aqueous polymer mature to form coated particles; and
(d) drying the coated particles obtained in step (c) to form the pharmaceutical composition wherein the amount of coating is less than 7% by weight of the pharmaceutical composition; and
(e) optionally filling the dried coated particles obtained in step (d) into capsules which can be opened by a patient and its contents sprinkled onto food to ease administration.

Preferably said active ingredient has a particle size of up to 50 microns, and is sensitive to heat and/or moisture, and/or has an unpleasant and/or bitter taste. More preferably, the active ingredient is an anticonvulsant agent, most preferably topiramate.

Surprisingly, is has been discovered that the amount of coating needed for satisfactory taste-masking and to preserve stability is low, i.e. less than 7% by weight of the pharmaceutical composition. This was unexpected in view of the coating levels disclosed in the prior art, especially in EP 1066027. The taste masking properties may be measured by the method disclosed in J. Pharm. Biomed. Anal. 29 (2002) pages 69-74.

Also included in the invention are methods of treating convulsions and epilepsy in a mammal in need thereof, which comprises administering to a mammal a therapeutically effective amount of any of those pharmaceutical compositions of the invention which comprise anti-convulsant drugs, preferably topiramate.

### DETAILED DESCRIPTION OF THE INVENTION

The present invention provides a solid pharmaceutical composition comprising preferably at least one drug which is sensitive to heat and/or moisture, and/or has an unpleasant and/or bitter taste, particularly an ant-convulsant drug, most preferably topiramate, said compositions being intended primarily for pediatric use, or for use in patients who cannot swallow whole tablets or capsules. More particularly, the solid pharmaceutical composition is a solid dosage formulation in the form of a sprinkle formulation comprising core particles comprising the active ingredient, and a taste mask coating.

The core particles may comprise at least one active ingredient and optionally one or more excipients.

The composition of the invention may comprise virtually any pharmaceutically active ingredient, or combination of active ingredients, except those which are chemically incompatible with the excipients and/or polymers used in the core particles and/or in the taste mask coating.

Preferably, the particle size of the active ingredient in the composition of the invention is up to 50 microns (μm), e.g. up to 40 microns (μm), for example up to 30 microns (μm), e.g. up to 20 microns (μm), e.g. up to 15 microns (μm). Experiments have shown that e.g. In the case where topiramate is the active ingredient, a particle size of up to 50 microns (μm) in the pharmaceutical composition according to the invention results in a product with the desired potency level.

The particles of the active ingredient in the composition of the invention may be processed as appropriate, e.g. ground and/or micronised according to conventional methods.

Preferably, the composition of the invention comprises at least one active ingredient which is sensitive to moisture and/or heat, and/or which has an unpleasant and/or bitter taste. Examples of bitter or unpleasant tasting drugs include, but are not limited to, e.g. antibiotics, including macrolides, such as erythromycin or darithromycin, penicillin, ampicillin, among others, as well as other active ingredients such as e.g. acetaminophen, caffeine, dextromethorpan, cimetidine, pseudoephedrine, diphenhydramine, spironolactone, chlorpheniramine, theophylline, and phenylbutazone, among others. Particularly, the composition of the invention comprises active ingredients which are sensitive to moisture and/or heat, and additionally have an unpleasant and/or bitter taste. Preferably, said active ingredients are anti-convulsant drugs, most preferably topiramate. As used herein, the term “topiramate” refers to compound 2,3:4,5-bis-O-(1-methylethylidene)-β-D-fructopyranose sulfamate (according to Merck index 2001-2003, Monograph number 09625).

The term “sensitive to heat and/or moisture” as used herein related to active ingredients is understood to mean that said active ingredients are subjected to a substantial degradation and/or decomposition upon exposure to heat and/or moisture during the manufacturing process and/or storage of said ingredients and/or of pharmaceutical compositions comprising said ingredients. Such active ingredients are known in the art.

Preferably the composition comprises a therapeutically effective amount of at least one active ingredient. The term “therapeutically effective amount” as used herein is understood to mean that amount of active ingredient which elicits the biological or medicinal response in a tissue, system, animal or human being that is being sought by a researcher, veterinarian, medical doctor or other clinician, which includes alleviation of the symptoms of the disease being treated.

The active ingredient is combined with excipients to provide core particles.

The term “excipients” as used herein, refers to any inert substance which may be combined with an active ingredient for preparing convenient dosage forms, including, for example, diluents, binders, lubricants, disintegrants, colors, flavors and sweeteners. Suitable diluents for use in the formulation and processes of the present invention include, but are not limited to, dicalclum phosphate, calcium sulfate, lactose, sorbitol, microcrystalline cellulose, kaolin, mannitol, sodium chloride, dry starch, powdered sugar and preferably sugar spheres.

The amount of the taste mask coating which is applied to the core particles, is preferably less than about 7% by weight, e.g. less than 7%, e.g. less than 6.5%, more preferably less than 6%, for example less than 5% by weight of the composition.

Preferably, the taste mask coating may constitute between about 1% and about 6% by weight, preferably between about 1% and about 5%, for example between about 1.5 and about 4%, optionally between about 2 and 3%, by weight of the composition.

In a preferred embodiment of the invention, a water dispersion comprising the active ingredient as defined above, preferably an anti-convulsant drug, and optionally a binder is sprayed onto sugar spheres and dried to provide core particles. Said dispersion is understood to mean any mixture wherein at least some or all of the anti-convulsant agent is dissolved, diluted, dispersed, suspended or emulsified in water. Suitable binders for use in the instant formulation and processes include, but are not limited to, synthetic gums such as hydroxypropyl methylcellulose, polyvinyl pyrrolidone (povidone), carboxymethylceliulose, ethylcellulose and methylcellulose, starch, pregelatnized starch, gelatin, sugars (e.g., molasses) and natural gums (e.g., acacia gum, sodium alginate, panwar gum). Preferably, povidone (especially, Povidone USP) is used as the binder. Coating and drying is preferably effected in a fluid bed coater of a Wurster type.

The core particles may then optionally be screened to remove fines and agglomerates.

The core partides are subsequently coated with an aqueous polymer mixture and then cured by conventional techniques. One suitable method of curing is drying.

Preferably, the aqueous polymer mixture is a taste masking mixture. The taste masking mixture, which is e.g. sprayed onto the core particles, comprises a taste masking agent dissolved, dispersed or suspended in water, and optionally at least one excipient. Preferably the taste masking agent comprises a polymer selected from an aminoalkyl methacrylate polymer or copolymer, or from ethyl cellulose. Most preferably, the taste masking agent is an aminoalkyl methacrylate polymer or copolymer. The excipient optionally used in the taste masking mixture may be selected from the excipients listed above, and may be e.g. triethyl citrate or talc, or a combination thereof.

Preferably, the amount of taste masking mixture is less than 7%, for example less than 6.5%, preferably less than 6%, for example less than 5% by weight of the composition. The taste masking mixture may constitute between about 1% and about 6% of the weight of the composition, preferably between about 1% and 5%, for example between about 1.5 and about 4%, optionally between about 2 and 3% of the weight of the composition.

The taste masking agent included in the taste masking mixture may constitute less than 6% by weight, e.g. less than 5% by weight of the composition, and may e.g. preferably range from about 1% to about 5%, e.g. from about 1.5% to about 4%, optionally between about 2 and 3%, by weight of the composition.

The coated particles may optionally be sifted to remove fines and agglomerates, and may subsequently be filled into capsules, e.g. sprinkle capsules which may be opened by the patients to sprinkle their contents onto food prior to consumption. The sprinkle capsules of the present invention are particularly advantageous with pediatric patients, who are prone to refuse unpleasant or bitter tasting medicaments. The sprinkle formulation of the invention allows for a good compliance with pediatric patients because the composition is palatable. If appropriate, patients, e.g. adult patients, may also swallow whole capsules containing the coated particles.

The most preferred process of the present invention comprises the following steps:
(a) preparing a water dispersion comprising topiramate and a binder;
(b) loading the dispersion obtained in step (a) onto sugar spheres in a fluid bed coater of a Wurster type;
(c) drying the loaded sugar spheres obtained in step (b);
(d) coating the dried loaded sugar spheres obtained in step (c) with an aqueous taste masking mixture to form coated particles; and
(e) curing the coated particles obtained in step (d), most preferably in a tray dryer and/or a fluid bed coater, to form the pharmaceutical composition wherein the amount of taste masking mixture is less than 7% by weight of the pharmaceutical composition; and
(f) filling the coated particles obtained in step (e) into capsules.

The term “particles” as used herein refers to free flowing substances of any shape which are larger than a powder including crystals, beads (smooth, round or spherical particles), spheres, e.g. sugar spheres, and granules.

The term “taste masking” as used herein refers to any substance, device or process which makes an oral pharmaceutical composition palatable and/or does not substantially release the active ingredient or agent in the mouth, but rather for example in the stomach or the intestinal tract.

The terms “aminoalkyl methacrylate polymer or copolymer” as used herein refer to copolymers of acrylic and methacrylic acid esters containing quaternary ammonium groups, and preferably refer to poly(ethyl acrylate-co-methyl methacrylate-co-trimethylammonloethyl methacrylate), most preferably to such substances described as “Ammonio Methacrylate Copolyrmer, Type A and B” of USP/NF. Examples of such substances are commercially available under the trademarks Eudragit® RL 30D and Eudragit® RS 30D, which are usually sold as e.g. 30% aqueous dispersions.

Ethyl cellulose may be purchased under the trademark Surelease®, e.g. as a dispersion.

A typical solid dosage pharmaceutical composition of the present invention is as follows:

| Ingredients | | |
|---|---|---|
| Core particles | | |
| Topiramate | 19-20% | w/w of composition |
| Sugar spheres | 65-70% | w/w of composition |
| Povidone K-30 | 9-10% | w/w of composition |
| Taste masking coat | | |
| Eudragit ® RL 30D or | 1-5% | w/w of composition |
| a combination of Eudragit ® RS | | |
| 30D + Eudragit ® RL 30D | | |
| (as dry polymer) | | |
| Triethyl citrate | <1% | w/w of composition |
| Talc | <1% | w/w of composition |

| | |
|---|---|
| Water | * |

*Essentially removed during drying

or alternatively, when ethyl cellulose is used:

| Ingredients | | |
|---|---|---|
| Core particles | | |
| Topiramate | 19-20% | w/w of composition |
| Sugar spheres | 65-70% | w/w of composition |
| Povidone K-30 | 9-10% | w/w of composition |
| Taste masking coat | | |
| Ethyl cellulose (Surerelease ® | 1-5% | w/w of composition |
| dispersion) (as dry polymer) | | |
| Triethyl citrate | <1% | w/w of composition |
| Talc | <1% | w/w of composition |

| | |
|---|---|
| Water | * |

*Essentially removed during drying

The dissolution profile of a typical composition of the invention as exemplified in Example 2 a measured in 500 ml phosphate buffer pH 6.8 with 0.1M Na Cl, with paddle mixing at 50 rpm, indicative of bloavailability for the compositions of the invention having less than 7% by weight taste mask coating, is shown in following table:

| Time (min) | % Release |
|---|---|
| 10 | ˜16 |
| 20 | ˜37 |
| 30 | ˜57 |
| 45 | ˜84 |
| 60 | ˜100 |

The composition of the invention show good stability when subjected to accelerated stress stability testing at 40° C. and at 75% relative humidity according to conventional methods, as exemplified in Example 4.

Additionally to methods of treating convulsions and/or epilepsy in a mammal in need thereof as described above, the present invention also includes methods of treating a condition selected from neuropathic pain, amyotrophic lateral sclerosis, acute ischemia, obesity, diabetes, psoriasis or bipolar disorder (including manic depression) in a mammal in need thereof which comprises administering to the mammal a therapeutically effective amount of those pharmaceutical composition of the inventions of the invention which comprise topiramate as active ingredient.

The following examples are provided to further define the invention without, however, limiting the invention.

### EXAMPLES

Polyvinyl pyrrolidone PVP K-30 and Topiramate are dispersed in water and disposed onto sugar spheres which are dried and coated with an aqueous dispersion of aminoalkyl methacrylate, talc and triethyl citrate; the resulting particles are cured and filled into capsules, which can be opened to sprinkle the composition onto food.

### Example

| | 1a | 1b | 1g |
|---|---|---|---|
| Ingredients | mg/capsule | mg/capsule | mg/capsule |
| Core | | | |
| Topiramate (micronised) | 50.00 | 25.00 | 15.00 |
| Povidone K-30 (PVPK-30) | 25.00 | 12.50 | 7.50 |
| Sugar spheres (710-850μ) | 175.00 | 87.50 | 52.50 |
| Purified water (as needed) | | | |
| Coating | | | |
| Eudragit ® RL 30D (solids) | 2.40 | 1.20 | 0.7 |
| Eudragit ® RS 30D (solids) | 5.40 | 2.70 | 1.60 |
| Talc | 2.34 | 1.17 | 0.7 |
| Triethyl citrate | 1.56 | 0.78 | 0.5 |
| Purified water (as needed) | | | |

### Example

| | 2a | 2b | 2c |
|---|---|---|---|
| Ingredients | mg/capsule | mg/capsule | mg/capsule |
| Core | | | |
| Topiramate (micronised) | 50.00 | 25.00 | 15.00 |
| Povidone K-30 (PVPK-30) | 25.00 | 12.50 | 7.50 |
| Sugar spheres (710-850μ) | 175.00 | 87.50 | 52.50 |
| Purified water (as needed) | | | |
| Coating | | | |
| Eudragit ® RL 30D (solids) | 4.00 | 2.00 | 1.20 |
| Eudragit ® RS 30D (solids) | 4.40 | 2.20 | 1.30 |
| Talc | 1.68 | 0.84 | 0.5 |
| Triethyl citrate | 1.68 | 0.84 | 0.5 |
| Purified water (as needed) | | | |

### Example 3

| Ingredients | mg/capsule |
|---|---|
| Core | |
| Topiramate (micronised) | 50.00 |
| Povidone K-30 (PVPK-30) | 25.00 |
| Sugar spheres (710-850μ) | 175.00 |
| Purified water (as needed) | |
| Coating | |
| Ethyl cellulose (solids) | 4.00 |
| (Surerelease ® dispersion) | |
| Talc | 1.68 |
| Triethyl citrate | 1.68 |
| Purified water (as needed) | |

### Example 4

Topiramate sprinkle capsules prepared according to Example 2a are subjected to stress stability testing at 40° C. relative humidity.

| Time (weeks) | Assay % |
|---|---|
| initial | 100.6 |
| 2 | 102.5 |
| 4 | 102.4 |
| 6 | 102.7 |
| 12 | 104.3 |

Assay % is understood to mean the potency of the drug as determined by HPLC. in particular, “Assay %” means contents of topiramate per capsule in %, determined at the points of time indicated above, relative to the theoretical initial nominal content of said capsule.

1. A pharmaceutical composition comprising
(a) core particles comprising a therapeutically effective amount of at least one active ingredient which is an anticonvulsant drug and optionally at least one excipient, and
(b) a taste mask coating, wherein the amount of taste mask coating is less than about 6.5 % by weight of the pharmaceutical composition.

2. The composition according to claim 1, wherein the active ingredient has a particle size of up to about 50 microns.

3. The composition according to claim 1, wherein the active ingredient is sensitive to heat, or moisture, or to both.

4. (canceled)

5. The composition according to claim 1, wherein the active ingredient comprises topiramate and the core particles further comprise at least one excipient.

6. The composition according to claim 5 wherein the core particles comprise topiramate as the active ingredient, a binder and a diluent.

7. The composition of claim 6 wherein the diluent comprises sugar spheres.

8. The composition according to claim 1 wherein the amount of taste mask coating is less than about 6% by weight of the pharmaceutical composition.

9. The composition according to claim 8 wherein the amount of taste mask coating ranges from about 1% by weight to about 5% by weight of the pharmaceutical composition.

10. (canceled)

11. The composition according to claim 1 wherein the taste mask coating comprises at least one aminoalkyl methacrylate polymer or copolymer or ethyl cellulose.

12-13. (canceled)

14. The composition according to claim 1 which comprises in the core particles from about 19% to about 20% topiramate, from about 65% to about 70% sugar spheres, from about 9% to about 10% polyvinyl pyrrolidone, and in the taste mask coating from about 1% to about 5% (as dry polymer) of aminoalkyl methacrylate polymer or copolymer, up to about 1% of triethyl citrate, and up to about 1% of talc, wherein % refers to percents by weight of the pharmaceutical composition.

15. A capsule comprising a pharmaceutical composition according to claim 1.

16. The capsule according to claim 15 in the form of a sprinkle capsule.

17. A process for preparing a pharmaceutical composition comprising the steps of:
(a) preparing core particles comprising at least one active ingredient which is an anticonvulsant drug;
(b) drying the core particles prepared in step (a) to form substantially dried core particles;
(c) coating the substantially dried core particles in step (b) with an aqueous polymer mixture to form coated particles; and
(d) drying the coated particles coated in step (c) to form the pharmaceutical composition wherein the amount of coating is less than about 6.5% by weight of the pharmaceutical composition; and
(e) optionally filling the dried coated particles obtained in step (d) into capsules.

18. A process according to claim 17 wherein the active ingredient has a particle size of up to about 50 microns.

19. A process according to claim 17 wherein the active ingredient is sensitive to heat or moisture, or to both, and which optionally is unpalatable.

20. The process according to claim 17 wherein the anti-convulsant drug comprises topiramate.

21. The process according to claim 17 wherein the amount of coating is less than about 6% by weight of the pharmaceutical composition.

22. The process according to claim 21 wherein the amount of coating ranges from about 1% by weight to about 5% by weight of the pharmaceutical composition.

23-27. (canceled)

28. A method of prevention or treatment of convulsions or epilepsy in a mammal comprising administering to said mammal a therapeutically effective amount of a pharmaceutical composition according claim 1.

29. (canceled)
